(19) United States
(12) Patent Application Publication
(10) Pub. No.: US 2010/0113918 A1
(43) Pub. Date: May 6, 2010

(54) SYSTEM AND METHOD FOR TRACKING OBJECT

(75) Inventor: Peter Traneus Anderson, Andover, MA (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/262,241

(22) Filed: Oct. 31, 2008

(51) Int. Cl. A61B 5/05

(52) U.S. Cl. 600/424

(57) ABSTRACT

In one embodiment, a position transponder for operation inside the body of a subject is provided. The transponder comprises a sensor coil and a magneto resistor coupled in series to the sensor coil.

### FIELD OF INVENTION

The invention generally relates to intrabody tracking systems and more particularly to methods and devices for tracking the position and orientation of an object in the body.

### BACKGROUND OF THE INVENTION

Many surgical, diagnostic, therapeutic and prophylactic medical procedures require the placement of objects such as sensors, treatment units, tubes, catheters, implants and other objects within the body.

In many instances, insertion of the object is for a limited time, such as during a surgery or catheterization. In other cases, objects such as feeding tubes or orthopedic implants are inserted for long-term use. A need exists for providing real-time information, for accurately determining the location and orientation of objects within a patient's body, while minimizing the use of X-ray imaging.

It is known in the art to use sensor coils as magnetic field transmitters and as magnetic field receivers. Further, the use of magnetic field sensors in determining the location and orientation of an object inside the patient's body is well known. Typically, the magnetic field sensor is located at the tip of a guidewire or a catheter and a plurality of leads connect the magnetic field sensor to an outside processing circuitry. The size of the magnetic field sensor located at the tip of the guidewire or the catheter is desired to be small and the number of leads connecting the magnetic field sensor to the outside processing circuitry is desired to be less.

Generally, a tracking system adapted for determining the location and orientation of an object, employs at least one magnetic field sensor, the at least one magnetic field sensor comprising a plurality of coils. A first coil provides five degrees of freedom (five location and orientation coordinates) and a second coil provides the sixth degree of freedom at the price of twice as many leads and twice as much space.

One of the prior art methods provides a magnetic field sensor using three co-located flux-gate magnetometers. A major disadvantage associated with this method is, the magnetic field sensor becomes bulky and employs a large number of leads thereby consuming more space and resource.

A number of other methods suggested in the prior art use three co-located coils and/or two non-coaxial coils (which may be co-located or positioned in Hazeltine configuration). This again is associated with a common disadvantage of using more space and resource.

Thus, there also exists a need for reducing the size of the magnetic field sensor used in tracking, as well as the number of leads used in the tracking system.

### BRIEF DESCRIPTION OF THE INVENTION

The above-mentioned shortcomings, disadvantages and problems are addressed herein which will be understood by reading and understanding the following specification.

In one embodiment, a position transponder for operation inside the body of a subject is provided. The transponder comprises a sensor coil and a magneto resistor coupled in series to the sensor coil.

In another embodiment, a position transponder for operation inside the body of a subject is provided. The transponder comprises a sensor coil, coupled so that a voltage drop is induced in the sensor coil responsive to one or more electromagnetic fields applied to the body in a vicinity of the transponder, a magneto resistor coupled to the sensor coil in series, such that a voltage drop is induced in the magneto resistor responsive to the electromagnetic fields applied to the body and a control unit coupled to the sensor coil and the magneto resistor so as to generate an output signal indicative of the voltage drop induced at the sensor coil and the voltage drop induced at the magneto resistor, such that the output signal is indicative of coordinates of the transponder inside the body. The control unit is further configured to transmit the output signal, so that the output signal is received by a signal processing unit positioned outside the body for use in determining the coordinates.

In yet another embodiment, a tracking system for tracking an object is provided. The tracking system comprises a radio frequency driver, adapted to transmit a radiofrequency driving current to the object, a plurality of transmitters adapted to generate electromagnetic fields at different respective frequencies in a vicinity of the object, a transponder coupled to the object and a signal processing unit coupled to the transponder. The transponder comprises a sensor coil, the sensor coil configured to sense a voltage drop in response to exposure to the electromagnetic fields and a magneto resistor coupled to the sensor coil in series, such that the magneto resistor is adapted to sense the electromagnetic field at a direction substantially perpendicular to the axis of the sensor coil and thereby experience a voltage drop. The transponder further comprises a control unit coupled to the sensor coil and the magneto resistor. The control unit is configured to generate and transmit an output signal, the output signal indicative of the voltage drop induced at the sensor coil and the voltage drop induced at the magneto resistor. Further, the signal processing unit is adapted to receive the output signal transmitted by the control unit and responsive thereto to determine the coordinates of the object.

In yet another embodiment, a method for tracking an object is provided. The method comprises positioning a radio frequency (RF) driver to transmit an RF driving current at a first frequency, to the object, coupling to the object a transponder comprising a sensor coil and a magneto resistor, driving a plurality of transmitters to generate electromagnetic fields at respective frequencies in a vicinity of the object that induce a voltage drop across the sensor coil and the magneto resistor, generating an output signal at the transponder indicative of the voltage drop across the sensor coil and the voltage drop across the magneto resistor, transmitting the output signal from the transponder and receiving and processing the output signal at a signal processing unit to determine coordinates of the object.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a block diagram of a transponder employed in a tracking system, in one embodiment;

FIG. 2 shows a block diagram of an intra-operative tracking system using the transponder shown at FIG. 1, in another embodiment;

FIG. 3 shows a schematic diagram of the intra-operative tracking system of FIG. 2 used in conjunction with an imaging system, in yet another embodiment; and

FIG. 4 shows a flow diagram depicting the method of tracking an object using the tracking system of FIG. 2.

### DETAILED DESCRIPTION OF THE INVENTION

In the following detailed description, reference is made to the accompanying drawings that form a part hereof, and in which is shown by way of illustration specific embodiments, which, may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

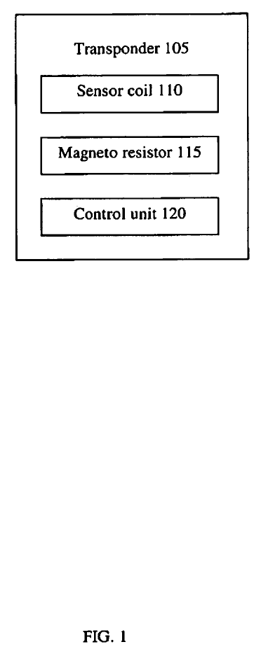

In one embodiment, shown in FIG. 1, a position transponder 105 for operation inside the body of a subject is provided. The transponder 105 comprises at least one sensor coil 110 and at least one magneto resistor 115 coupled in series to the sensor coil 110. One or more electromagnetic fields are applied to the body in a vicinity of the transponder 105. The application of electromagnetic fields induces a voltage drop in each of the sensor coil 110 and the magneto resistor 115. The transponder 105 further comprises a control unit 120, coupled to the sensor coil 110 and the magneto resistor 115 so as to generate an output signal indicative of the voltage drop induced at the sensor coil 110 and the voltage drop induced at the magneto resistor 115. The output signal is indicative of coordinates of the transponder 105 inside the body. The control unit 120 is further configured to transmit the output signal to a signal processing unit positioned outside the body, such that the output signal is received by the signal processing unit for use in determining the coordinates of the transponder 105.

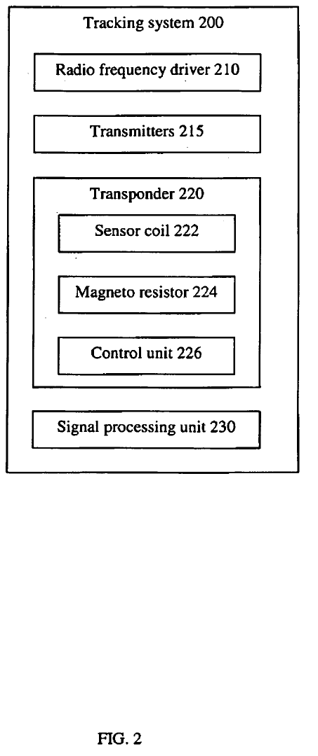

In practice, the transponder 105 is tracked against a plurality of transmitters. The plurality of transmitters emit at different respective frequencies including a second frequency. Further, a radiofrequency driver is configured to drive the transponder 105 with a sine wave at a first frequency. This is further explained in conjunction with FIG. 2.

Accordingly, in one embodiment, as shown in FIG. 2, a tracking system 200 for tracking, an object (not shown) is provided. The tracking system 200 comprises a radio frequency driver 210, adapted to transmit a radiofrequency driving current, to the object (not shown) via one or more connecting leads connecting the transponder 105 to an outside circuitry comprising the radio frequency driver 210, a plurality of transmitters 215 adapted to generate electromagnetic fields at different respective frequencies in a vicinity of the object (not shown), a transponder 220 coupled to the object (not shown) and a signal processing unit 230 coupled to the transponder 220.

The plurality of transmitters 215 generate electromagnetic fields composed of a plurality of differently oriented field components each having a different known frequency in the range of 2-10 kHz. Each of these field components are sensed by each of the sensor coil 222 and the magneto resistor 224 which each produce a signal comprising one or more frequency components having different amplitudes and phases depending on the relative distance and orientation of the particular sensor coil 222 or the magneto resistor 224 from the particular transmitter which transmits a particular frequency. The contributions of each of the transmitters 215 are used to solve a set of field equations, which are dependent upon the field form. Solving these equation sets produces the location and orientation of the transponder 220.

The transponder 220 is typically about 2-5 mm in length and about 2-3 mm in outer diameter, enabling it to fit conveniently inside the object (not shown). The sensor coil 222 is optimized to receive and transmit high-frequency signals, in the range of 1 MHz. However, the sensor coil 222 is designed for operation in the range of 1-3 kHz, the frequencies at which the transmitters 215 generate the electromagnetic fields. Alternatively, other frequency ranges may be used, as dictated by application requirements.

The sensor coil 222 in the transponder 220 has an inner diameter, of about 0.5 mm and has approximately 800 turns of about 16 micrometer diameter to provide an overall diameter in the range of 1-1.2 mm. Skilled artisans shall however appreciate that these dimensions may vary over a considerable range and are only representative of a range of dimensions. The effective capture area of the sensor coil 222 is about 400 mm.sup.2. The effective capture area is desired be made as large as feasible, consistent with the overall size requirements. Though the shape of the sensor coil 222 used in one embodiment is cylindrical, other shapes can also be used depending on the geometry of the object (not shown). An example of the sensor coil 222 is the T30AA01 passive telecoil manufactured by the Sonion division of Pulse Engineering.

The electromagnetic fields produced by the transmitters 215 induce a voltage drop in the sensor coil 222. The voltage drop at the sensor coil 222 comprises a component at the second frequency, the frequency of the electromagnetic fields produced by the transmitters 215. The voltage components are proportional to the strengths of the components of the respective magnetic fields produced by the transmitters 215 in a direction parallel to the axis of the sensor coil 222. Thus, the amplitudes of the voltages indicate the position and orientation of the sensor coil 222 relative to the fixed transmitters 215.

The magneto resistor 224 is coupled to the sensor coil 222 in series using one of a single twisted-pair and a coaxial cable, such that the magneto resistor 224 is adapted to sense the electromagnetic field at a direction substantially perpendicular to the axis of the sensor coil 222. This configuration is aimed at minimizing the field coupling between the sensor coil 222 and the magneto resistor 224.

An example of the magneto resistor 224 is an extraordinary magneto resistance (EMR) device. Extraordinary magneto resistance (EMR) devices have been fabricated and characterized at various magnetic fields, operating temperatures, and current excitations. The extraordinary magneto resistance devices are comprised of nonmagnetic high mobility semiconductors and low resistance metallic contacts and shunts. The resistance of the extraordinary magneto resistance device is modulated by magnetic fields due to the Lorentz force steering an electron current between a high resistance semiconductor and a low resistance metallic shunt.

The magneto resistor 224 comprises a first portion, where the resistance does not significantly change with the electromagnetic field. Therefore, the voltage drop at the magneto resistor 224 comprises a component at the first frequency, the frequency of the driving currents flowing through the transmitters 215.

On the other hand, the magneto resistor 224 comprises a second portion, where the electrical resistance of the magneto resistor 224 varies responsive to the changing electromagnetic field. Following Ohm's law, V=IR, the magneto resistor 224 develops a voltage drop that varies with the product of the applied electromagnetic field and the current through the magneto resistor 224. As the driving current is at the first frequency, with a zero direct current component, and the electromagnetic field is at the second frequency, the voltage drop at the magneto resistor 224 comprises components at the sum of the first frequency and the second frequency and at the difference between the first frequency and the second frequency

As the voltage drops induced at the sensor coil 222 and the magneto resistor 224 due to the electromagnetic field are at different frequencies, the two voltage drops can be distinguished when measuring their sum through two connecting leads.

The control unit 226 coupled to the sensor coil 222 and the magneto resistor 224 comprises suitable circuitry for reading the signals from the sensor coil 222 and the magneto resistor 224. For example, in one embodiment, the control unit 226 comprises at least one of a balanced bridge and hybrid-circuit electronics to read the signals, in the presence of the signal from the radio frequency driver 210. Skilled artisans shall however appreciate other suitable circuits and methods for signal processing.

Responsive to reading the signals from the sensor coil 222 and the magneto resistor 224, the control unit 226 generates an output signal indicative of an amplitude of the voltage drop induced at the sensor coil 222, an amplitude of the voltage drop induced at the magneto resistor 224 and a phase of the voltage drop relative to a phase of the electromagnetic fields. The signal processing unit 230 is adapted to determine the coordinates and an orientation of the object (not shown), responsive to the amplitude and the phase of the voltage drop indicated by the output signal.

Skilled artisans shall however appreciate that both analog and digital embodiments of signal processing are possible. The signal processing unit 230 represents an assemblage of units to perform intended functions. For example, such units may receive information or signals, process information, function as a controller, display information, and/or generate information or signals. Typically the signal processing unit 230 may comprise one or more microprocessors.

The transponder 220, as described above, can be employed to provide all six position and orientation coordinates (X, Y, Z yaw, pitch and roll) of the object (not shown). The single sensor coil 222 shown in FIG. 2, in conjunction with one or more transmitters 215, enables the signal processing unit 230 to generate three dimensions of position and two dimensions of orientation information. The third dimension of orientation (typically rotation of the object (not shown) about its longitudinal axis) can be inferred from the magneto resistor 224. Although the signal from the magneto resistor 224 is smaller than the signal from the sensor coil 222, the signal from the magneto resistor 224 is large enough to provide the roll information.

The description above primarily concerns with acquiring information by a set of a sensor coil 222 and a magneto resistor 224, used to determine the position and orientation of a remote object (not shown) such as a medical device or instrument. It is also within the scope of the invention that the transponder 220 may comprise more than one set of sensor coils or magneto resistors that will provide sufficient parameters to determine the configuration of the remote object (not shown), relative to a reference frame.

Accordingly, in one embodiment, one or more magneto resistors can be combined with one or more sensor coils to obtain six position and orientation coordinates for the object (not shown). For example, a plurality of magneto resistors can be used along with one or more sensor coils or a plurality of sensor coils can be used along with one or more magneto resistors to form a transponder 220. Further, each magneto resistor 224 can be connected to a single sensor coil 222 using a single pair of leads

In an alternative embodiment, the transponder 220 can be tracked against a plurality of receivers. Accordingly, the tracking system 200 can comprise a plurality of receivers and the sensor coil 222 can be selected to be a five degree of freedom (“5DOF”) sensor. Further, similar to the tracking system 200 described above, the magneto resistor 224 can be employed to provide the roll information

In yet another alternative embodiment, the transponder 220 can be tracked against an array comprising at least one transmitter and at least one receiver. Further, each receiver can comprise a magnetic field sensor such as but not limited to a magneto resistor 224.

The tracking system 200 described in various embodiments can be used as a part of a surgical navigation product. For this application, the transponder 220 is adapted to be inserted, together with the object (not shown), into the body of the subject, while one or more transmitters 215 and the RF driver 210 are placed outside the body.

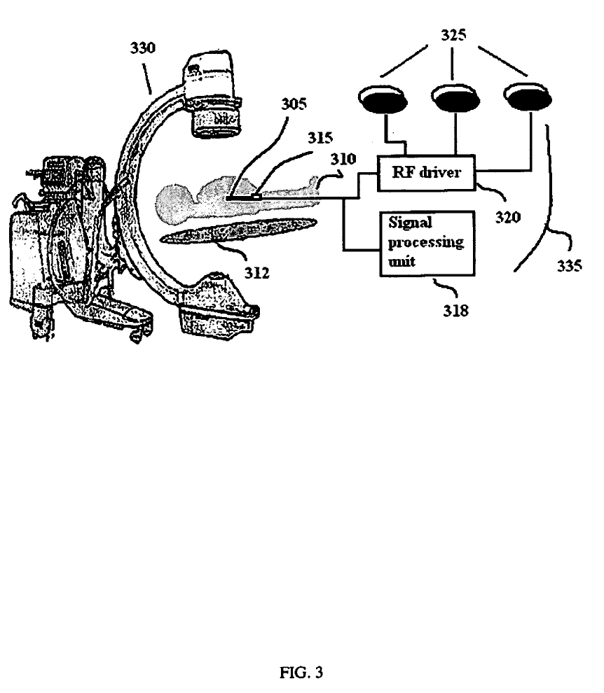

In an exemplary embodiment, shown at FIG. 3, an object 305 includes an elongate probe, for insertion into the body of a subject 310 positioned on a patient positioning system 312. A transponder 315 is fixed to the probe so as to enable an externally located signal processing unit 318 to determine the coordinates of a distal end of the probe. Alternatively, the object 305 includes an implant, and the transponder 315 is fixed in the implant so as to enable the signal processing unit 318 to determine the coordinates of the implant within the body. Further, the transponder 315 may be fixed to other types of invasive tools, such as endoscopes, catheters and feeding tubes, as well as to other implantable devices, such as orthopedic implants.

An externally-located radio frequency driver 320 sends a radio frequency (RF) signal, having a frequency in the kilohertz range, to drive the transponder 315. Additionally, a plurality of electromagnetic transmitters 325 positioned in fixed locations outside the body produce electromagnetic fields at different, respective frequencies, typically in the kilohertz range. These fields induce voltage in the sensor coil 222 and the magneto resistor 224 of the transponder 315, which depend on the spatial position and orientation of the sensor coil 222 and the magneto resistor 224 relative to the transmitters 325. The control unit 226 converts the voltages into high-frequency signals, which are transmitted by the control unit 226, in the form of output signal, to the externally-located signal processing unit 318. The signal processing unit 318 processes the output signal to determine the position and orientation coordinates of the transponder 315, for display and recording.

Typically, prior to performing a medical procedure, the image of the subject 310 is captured using an imaging device 330 (such as an X-ray imaging device) and is displayed on a computer monitor. The transponder 315 is visible in the X-ray image, and the position of the transponder 315 in the image is registered with the respective location coordinates, as determined by the signal processing unit 318. During the medical procedure, the movement of the transponder 315 is tracked by the tracking system 335 and is used to update the position of the transponder 315 in the image on the computer monitor, using image processing techniques known in the art. The updated image can be used to achieve desired navigation of the object 305 during the medical procedure, without the need for repeated X-ray exposures during the medical procedure.

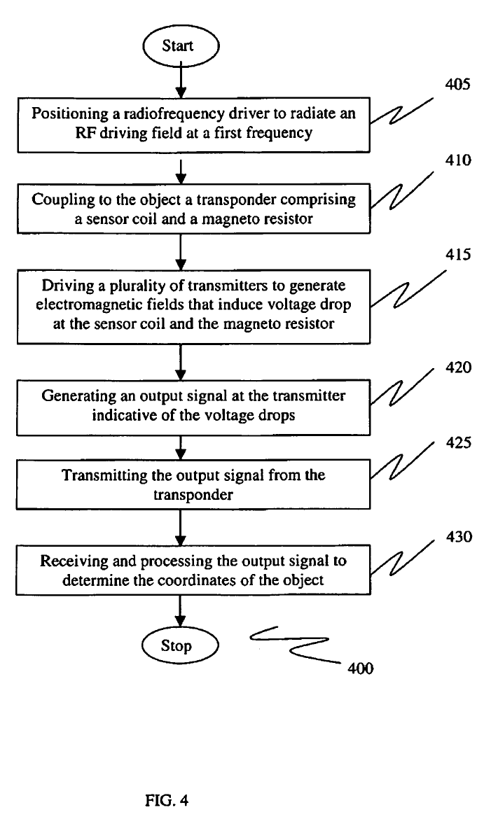

In another embodiment shown at FIG. 4, a method 400 for tracking an object 305 is provided. The method 400 comprises positioning a radio frequency (RF) driver 320 to transmit an RF driving current to the object 305 step 405, coupling to the object 305 a transponder 315 comprising a sensor coil 222 and a magneto resistor 224 step 410, driving a plurality of transmitters 325 to generate electromagnetic fields at respective frequencies in a vicinity of the object 305 that induce a voltage drop across the sensor coil 222 and the magneto resistor 224 step 415, generating an output signal at the transponder 315 indicative of the voltage drop across the sensor coil 222 and the voltage drop across the magneto resistor 224 step 420, transmitting the output signal from the transponder 315 step 425 and receiving and processing the output signal at the signal processing unit 318 to determine coordinates of the object 305 step 430.

In some embodiments, the method 400 includes inserting the transponder 315, together with the object 305, into the body of the subject 310. Further, positioning the plurality of the transmitters 325 and the RF driver 320 includes placing one or more transmitters 325 and the RF driver 320 outside the body.

In an exemplary embodiment, to operate the transponder 315, the subject 310 is placed in a magnetic field generated, for example, by situating under the subject 310 a pad containing the plurality of transmitters 325 for generating the electromagnetic field. The plurality of transmitters 325 generate electromagnetic fields at different, respective frequencies. A reference electromagnetic field sensor (not shown) is fixed relative to the subject 310, for example, taped to the back of the subject 310, and the object 305 with the transponder 315 coupled thereto is advanced into the body of the subject 310. Signals received from the transponder 315 are conveyed to the signal processing unit 318, which analyzes the signals and then displays the results on a monitor. By this method, the precise location of transponder 315, relative to the reference sensor (not shown), can be ascertained and visually displayed. Furthermore, the reference sensor (not shown) may be used to correct for breathing motion or other movement in the subject 310. In this way, the acquired position and orientation of the object 305 may be referenced to an organ structure and not to an absolute outside the reference frame, which is less significant.

As described in various embodiments, the invention combines a sensor coil 222 with a magneto resistor 224 to obtain a transponder 220. The magneto resistor 224 replaces a second sensor coil typically employed in prior art systems, thereby eliminating the use of the second sensor coil. A major advantage associated with the magneto resistor 224 is its ability to be fabricated as a miniature device. Thus, replacing the second sensor coil with a magneto resistor 224 smaller than the second sensor coil reduces the space needed.

Further, the magneto resistor 224 and the sensor coil 222 can share a single pair of leads. Thus, using the magneto resistor 224, allows for a simplified guidewire fabrication as the number of leads employed in connecting two components is reduced by half. Thus, the use of the magneto resistor 224 in the transponder 220 enables the transponder 220 to obtain six degrees of freedom (“6DOF”) without causing much burden on resource or space.

In various embodiments, system and method for tracking an object are described. However, the embodiments are not limited and may be implemented in connection with different applications. The application of the invention can be extended to other areas. For example, in cardiac applications such as in catheter or flexible endoscope for tracking the path of travel of the catheter tip, to facilitate laser eye surgery by tracking the eye movements, in evaluating rehabilitation progress by measuring finger movement, to align prostheses during arthroplasty procedures and further to provide a stylus input for a Personal Digital Assistant (PDA). The invention provides a broad concept of tracking an object in obscure environment, which can be adapted to track the position of items other than medical devices in a variety of applications. That is, the tracking system may be used in other settings where the position of an object in an environment is unable to be accurately determined by visual inspection. For example, tracking technology may be used in forensic or security applications. Retail stores may use tracking technology to prevent theft of merchandise. Tracking systems are also often used in virtual reality systems or simulators. Accordingly, the invention is not limited to a medical device. The design can be carried further and implemented in various forms and specifications.

This written description uses examples to describe the subject matter herein, including the best mode, and also to enable any person skilled in the art to make and use the subject matter. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A position transponder for operation inside the body of a subject, the transponder comprising:
a sensor coil; and
a magneto resistor coupled in series to the sensor coil.

2. The transponder of claim 1, wherein the sensor coil is adapted to sense a voltage drop in response to one or more electromagnetic fields applied to the body in a vicinity of the transponder.

3. The transponder of claim 2, wherein the sensor coil is coupled to the magneto resistor via one of a single twisted pair or a coaxial cable.

4. The transponder of claim 3, wherein the magneto resistor is adapted to sense the electromagnetic fields at a direction substantially perpendicular to the axis of the sensor coil and thereby experience a voltage drop.

5. The transponder of claim 4, further comprising a control unit coupled to the sensor coil and the magneto resistor so as to generate an output signal indicative of the voltage drop induced at the sensor coil and the voltage drop induced at the magneto resistor, such that the output signal is indicative of coordinates of the transponder inside the body.

6. The transponder of claim 5, wherein the control unit is further configured to transmit the output signal, so that the output signal is received by a signal processing unit positioned outside the body for use in determining the coordinates.

7. The transponder of claim 6, wherein the control unit comprises a balanced bridge or hybrid circuit electronics.

8. The transponder of claim 6, wherein the control unit is adapted to generate the output signal indicative of an amplitude of the voltage drop and a phase of the voltage drop, and wherein the signal processing unit is adapted to determine the coordinates and an orientation of the object, responsive to the amplitude and the phase of the voltage drop indicated by the output signal.

9. A position transponder for operation inside the body of a subject, the transponder comprising:
a sensor coil, coupled so that a voltage drop is induced in the sensor coil responsive to one or more electromagnetic fields applied to the body in a vicinity of the transponder;
a magneto resistor coupled to the sensor coil in series, such that a voltage drop is induced in the magneto resistor responsive to the electromagnetic fields applied to the body; and
a control unit, coupled to the sensor coil and the magneto resistor so as to generate an output signal indicative of the voltage drop induced at the sensor coil and the voltage drop induced at the magneto resistor, such that the output signal is indicative of coordinates of the transponder inside the body.

10. The transponder of claim 9, wherein the sensor coil is coupled to the magneto resistor via one of a single twisted pair or a coaxial cable.

11. The transponder of claim 9, wherein the magneto resistor is adapted to sense the electromagnetic field at a direction substantially perpendicular to the axis of the sensor coil.

12. The transponder of claim 9, wherein the control unit is further adapted to transmit the output signal, so that the output signal is received by a signal processing unit positioned outside the body for use in determining the coordinates.

13. The transponder of claim 12, wherein the control unit is adapted to generate the output signal indicative of an amplitude of the voltage drop and a phase of the voltage drop, and wherein the signal processing unit is adapted to determine the coordinates and an orientation of the object, responsive to the amplitude and the phase of the voltage drop indicated by the output signal.

14. The transponder of claim 13, wherein the control unit comprises a balanced bridge or hybrid circuit electronics.

15. A tracking system for tracking an object comprising:
a radio frequency driver, adapted to transmit a radiofrequency driving current, at a first frequency, to the object;
a plurality of transmitters adapted to generate electromagnetic fields at different respective frequencies, including a second frequency, in a vicinity of the object;
a transponder coupled to the object, the transponder comprising:
a sensor coil, the sensor coil configured to sense a voltage drop in response to exposure to the electromagnetic fields;
a magneto resistor coupled to the sensor coil in series, such that the magneto resistor is adapted to sense the electromagnetic field at a direction substantially perpendicular to the axis of the sensor coil and thereby experience a voltage drop; and
a control unit coupled to the sensor coil and the magneto resistor, so as to generate an output signal indicative of the voltage drop induced at the sensor coil and the voltage drop induced at the magneto resistor; and
a signal processing unit coupled to the transponder, the signal processing unit adapted to receive the output signal transmitted by the control unit and responsive thereto to determine the coordinates of the object.

16. The tracking system of claim 15, wherein the sensor coil is coupled to the magneto resistor via one of a single twisted pair or a coaxial cable.

17. The tracking system of claim 15, wherein the control unit comprises a balanced bridge or hybrid circuit electronics.

18. The tracking system of claim 15, wherein the output signal is analog.

19. The tracking system of claim 15, wherein the output signal is digital.

20. The tracking system of claim 15, wherein the object is a catheter or an endoscope.

21. The tracking system of claim 15, wherein the control unit is adapted to generate the output signal indicative of an amplitude of the voltage drop and a phase of the voltage drop, and wherein the signal processing unit is adapted to determine the coordinates and an orientation of the object, responsive to the amplitude and the phase of the voltage drop indicated by the output signal.

22. A method for tracking an object, comprising:
positioning a radio frequency (RF) driver to transmit an RF driving current at a first frequency, to the object;
coupling to the object a transponder comprising a sensor coil and a magneto resistor;
driving a plurality of transmitters to generate electromagnetic fields at respective frequencies in a vicinity of the object that induce a voltage drop across the sensor coil and the magneto resistor;
generating an output signal at the transponder indicative of the voltage drop across the sensor coil and the voltage drop across the magneto resistor;
transmitting the output signal from the transponder; and
receiving and processing the output signal to determine coordinates of the object.

23. The method of claim 22, wherein driving the plurality of transmitters comprises driving the plurality of transmitters to generate the electromagnetic fields at different respective frequencies including a second frequency.

24. The method of claim 22, further comprising inserting the transponder, together with the object, into the body of a subject.

25. The method of claim 22, wherein positioning the plurality of transmitters and the RF driver comprises placing the plurality of transmitters and the RF driver outside the body.
